(19) United States
(12) Patent Application Publication
(10) Pub. No.: US 2013/0229504 A1
(43) Pub. Date: Sep. 5, 2013

(54) THREE DIMENSIONAL ULTRASONIC GUIDANCE OF SURGICAL INSTRUMENTS

(71) Applicant: Jeanne Cheng, Andover, MA (US)

(72) Inventor: Jeanne Cheng, Andover, MA (US)

(73) Assignee: KONINKLIJKE PHILIPS ELECTRONICS N.V., EINDHOVEN (NL)

(21) Appl. No.: 13/883,780

(22) PCT Filed: Nov. 10, 2011
(86) PCT No.: PCT/IB2011/055018

§ 371 Date: May 7, 2013

(30) Foreign Application Priority Data
Nov. 19, 2010 (US) 61/415644

(51) Int. Cl. A61B 1/00

(52) U.S. Cl. CPC A61B 1/00009
USPC 348/65

(57) ABSTRACT

An ultrasonic imaging system is used to observe and guide insertion of a needle into the body to access a targeted surgical site. A two dimensional array probe scans a volumetric region including the surgical site and a multiplanar reformatter formats the resulting 3D echo dataset to form a sequence of spatially adjacent images in real time. A plurality of the spatially adjacent images are concurrently displayed in real time. As the clinician inserts the needle into the body its progress of insertion may be observed in one plane. But if the insertion path of the needle is not constrained to one plane but passes through numerous planes, the insertion path is seen in successive ones of the concurrently displayed adjacent images.

This invention relates to ultrasonically guided invasive procedure and, in particular, to invasive procedures guided by three dimensional ultrasonic imaging.

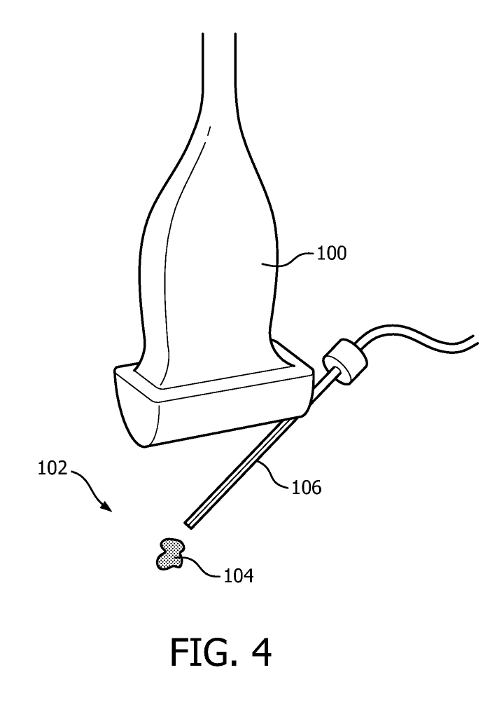

A number of surgically invasive procedures can be guided by ultrasonic imaging, which displays the interior of the tissue which is the subject of the invasive procedure. Predominate among such procedures are those requiring needle guidance and targeting, such as biopsies of observed masses in the breast and regional anesthesia administration. In these procedures the target tissue can be visualized with ultrasound as well as the path of the needle as it passes through tissue toward the target tissue. A number of ultrasound imaging systems and devices have been developed for the performance of such procedures. When two dimensional (2D) ultrasound imaging is used, it is important to keep the needle aligned with the image plane. This is illustrated in FIG. 4, which shows an ultrasound probe 100 that scans a 2D image plane 102. The probe is positioned so that the target tissue 104 is visible in the image. The needle 106 accessing the target tissue 104 must continually travel in the image plane 102. If the needle travels out of the image plane it can no longer be visualized and observed as it approaches the target tissue. Biopsy guides are commercially available for many ultrasound probes which allow the needle to be introduced into the body only in the plane of the ultrasound image. Another technique for dealing with this requirement is described in U.S. Pat. No. 5,158,088 (Nelson et al.). In the Nelson et al. system, a transducer is located at the tip of the introducer stylet, which broadcasts a signal that is received by the ultrasonic imaging probe. This signal is received by the probe and used to develop an audible signal as the tip of the stylet approaches and then intersects the imaging plane. Signals received by the stylet transducer can be used to identify the tip of the stylet in the 2D ultrasound image. Another 2D imaging technique is described in U.S. Pat. No. 5,095,910 (Powers). The Powers system vibrates the stylet and this vibratory motion is detected by ultrasonic Doppler techniques. The color Doppler signal in the ultrasound image indicates the location of the tip of the stylet. But again, the stylet must be in the image plane in order for

Doppler detection and imaging to occur.

Three dimensional (3D) ultrasonic imaging has shown promise in overcoming the 2D image plane alignment problem. Since 3D imaging images a volume of tissue and not just a single plane, the constraint of alignment with a single plane is avoided. But many clinicians are not familiar with 3D ultrasound or the appearance of anatomy in 3D ultrasonic images. In addition, surround tissue can obscure the target tissue, the needle in the imaged volume, or both. U.S. Pat. No. 7,529,393 (Peszynski et al.) shows several approaches to dealing with these difficulties, including displaying the tip of the needle with greater display line density, showing the needle tip in a smaller subvolume, and combining both 2D and 3D imaging in one display. Another way to use 3D imaging is to display three mutually orthogonal image planes which converge at the tip of the surgical instrument, as described in U.S. Pat. No. 6,572,547 (Miller et al.) and U.S. Pat. pub. no. US2010/0121190. Yet a third way is described for computed tomography and CT fluoroscopy in U.S. Pat. pub. no. US2007/0100234 (Arenson et al.) In the Arenson et al. system a fan beam of x-rays is projected toward several rows of detector elements. Each row of detectors is used to reconstruct an image, and all of the rows are used for multi-slice CT fluoroscopic imaging. When the needle passes through the tissue imaged by the multiple slices, the needle is detected in each image and the multiple images are combined to form a composite thick slice image shows all of the needle segments in all of the combined slice images. However the patient table or the gantry must be constantly adjusted to keep the target tissue in line between the x-ray source and the detectors. In addition, fluoroscopy exposes the patient and the operator to ionizing radiation. Accordingly it is desirable to provide an ultrasonic technique for surgical instrument guidance so as to avoid ionizing radiation. It is further desirable for the ultrasonic technique to avoid the problem of image plane and needle alignment that is faced by prior art techniques, and to provide a system that is simple to use and readily comprehended by those not well familiar with 3D ultrasound imaging.

In accordance with the principles of the present invention, an ultrasonic imaging system and method are described for guiding an invasive instrument such as a surgical needle to target tissue in the body. The system uses a probe with a two dimensional array of transducer elements which electronically steers beams in three dimensions so as to scan a volumetric region of the body in real time. The 2D array probe can be easily manipulated to acquire images of the target tissue and the path traveled by an invasive device to reach the target tissue, and to optimize the angle of incidence between the ultrasound beams and the instrument. The echoes received from three dimensions of tissue are processed by a multiplanar reformatter into a plurality of spatially adjacent 2D image planes. The images of the spatially adjacent planes are concurrently display in the sequence of their spatial order in the tissue and continually updated in real time. As the invasive device approaches the target tissue its passage can be followed from one image plane to the next, and the spatial order of the images gives the clinician an intuitive sense of the progress of the instrument travel. Adjacent images can overlap each other in the thickness dimension so that the needle can be seen in adjacent images simultaneously and its insertion progress more easily followed.

In the drawings:

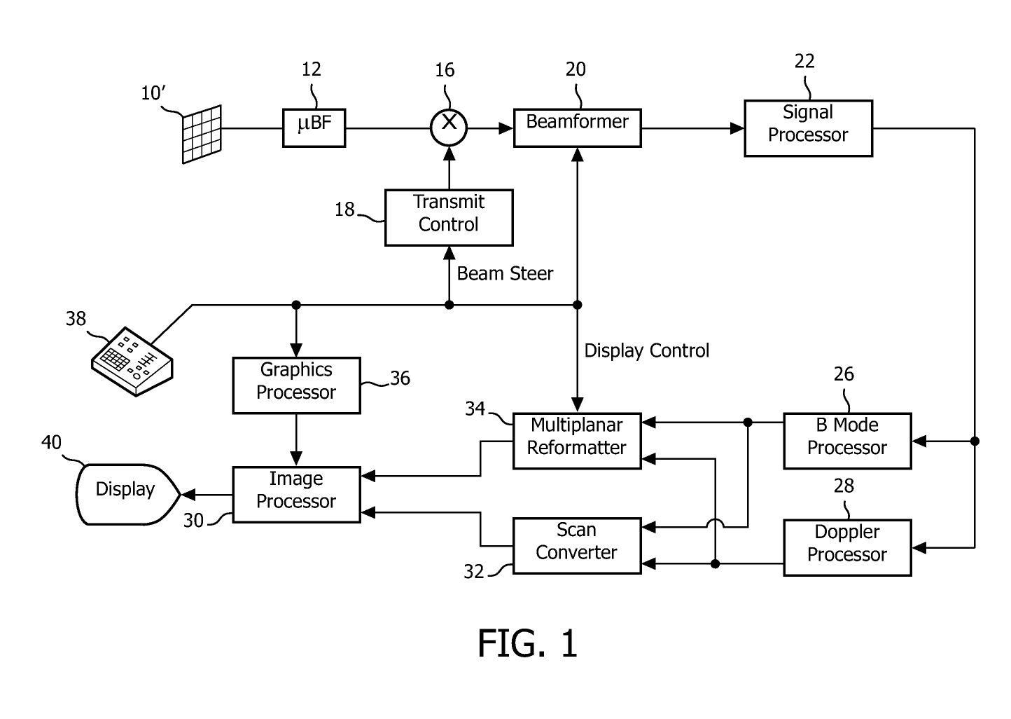

FIG. 1 illustrates in block diagram form an ultrasonic diagnostic imaging system constructed in accordance with the principles of the present invention.

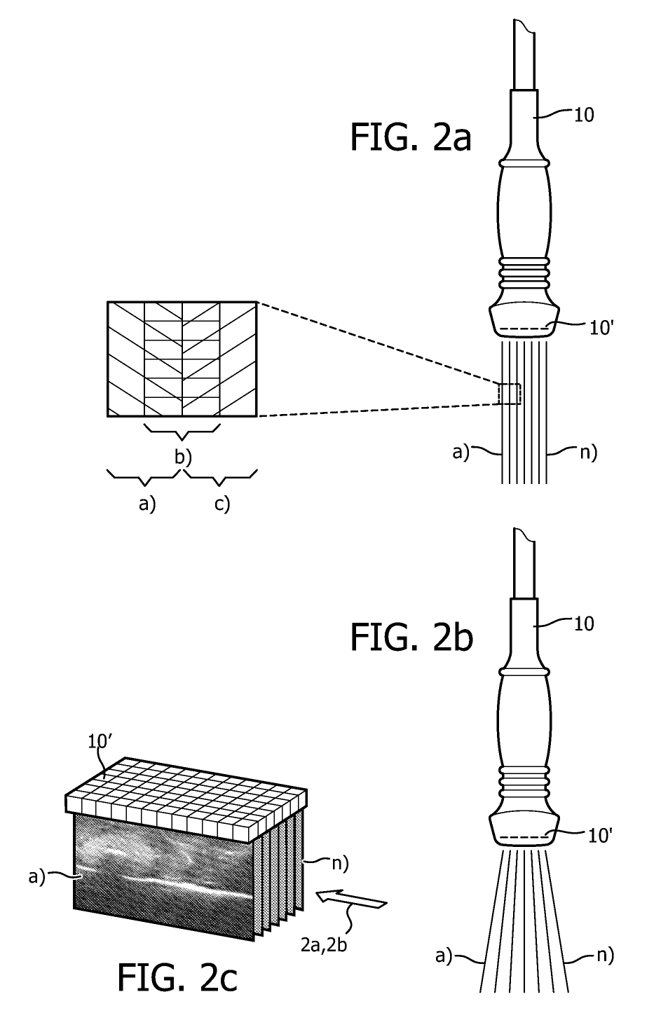

FIGS. 2a and 2b illustrate different planar alignments which may be produced by an ultrasound probe of the present invention and overlapping thick slice image planes.

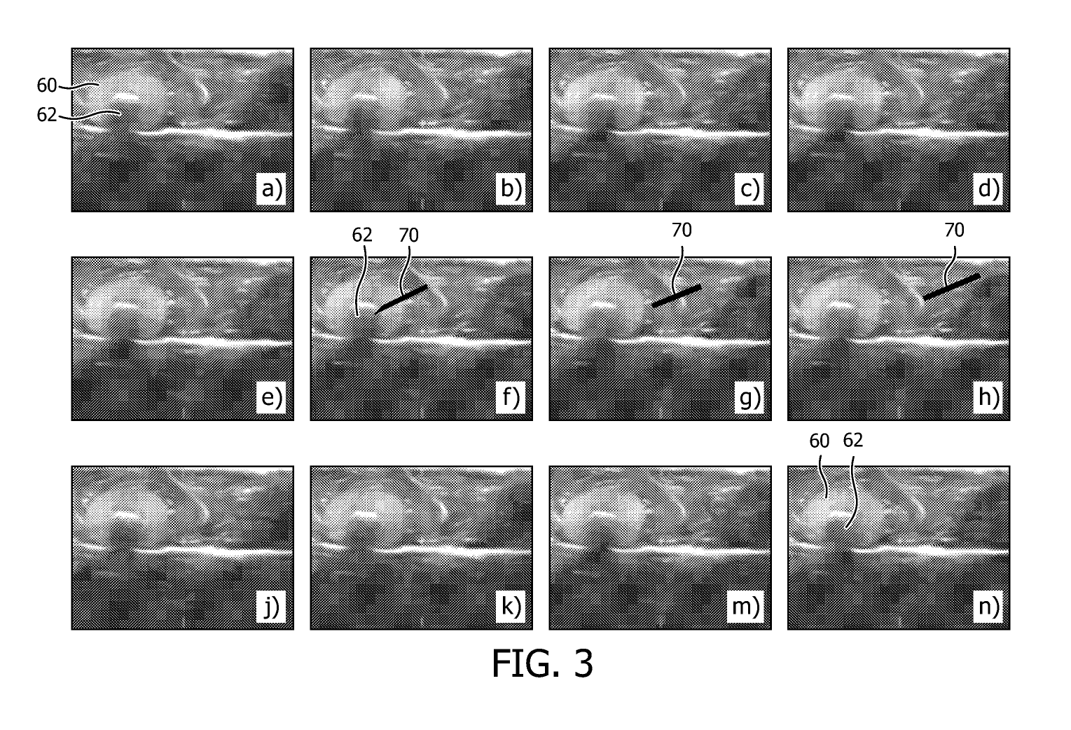

FIG. 3 illustrates a sequential display of spatially adjacent image planes of a needle in tissue in accordance with the principles of the present invention.

FIG. 4 illustrates the introduction of a needle in the two dimensional image plane of an ultrasound probe.

Referring first to FIG. 1, an ultrasonic diagnostic imaging system constructed in accordance with the principles of the present invention is shown in block diagram form. In FIG. 1 a transducer array 10′ is provided in an ultrasound probe 10 for transmitting ultrasonic waves and receiving echo information. The transducer array 10′ is a two dimensional array of transducer elements capable of scanning in three dimensions for 3D imaging. The transducer array is coupled to a microbeamformer 12 in the probe which controls transmission and reception of signals by the array elements. Microbeamformers are capable of at least partial beamforming of the signals received by groups or “patches” of transducer elements as described in U.S. Pat. No. 5,997,479 (Savord et al.), No. 6,013,032 (Savord), and No. 6,623,432 (Powers et al.) The microbeamformer is coupled by the probe cable to a transmit/receive (T/R) switch 16 which switches between transmission and reception and protects the main beamformer 20 from high energy transmit signals. The transmission of ultrasonic beams from the transducer array 10 under control of the microbeamformer 12 is directed by the transmit controller 18 coupled to the T/R switch and the beamformer 20, which receives input from the user's operation of the user interface or control panel 38. One of the functions controlled by the transmit controller is the direction in which beams are steered. Beams may be steered straight ahead from (orthogonal to) the transducer array, or at different angles for a wider field of view as described below.

The partially beamformed signals produced by the microbeamformer 12 are coupled to a main beamformer 20 where partially beamformed signals from the individual patches of elements are combined into a fully beamformed signal. For example, the main beamformer 20 may have 128 channels, each of which receives a partially beamformed signal from a patch of 12 transducer elements. In this way the signals received by over 1500 transducer elements of a two dimensional array can contribute efficiently to a single beamformed signal.

The beamformed signals are coupled to a signal processor 22. The signal processor 22 can process the received echo signals in various ways, such as bandpass filtering, decimation, I and Q component separation, and harmonic signal separation which acts to separate linear and nonlinear signals so as to enable the identification of nonlinear echo signals returned from tissue and microbubbles. The signal processor may also perform additional signal enhancement such as speckle removal, signal compounding, and noise elimination.

The processed signals are coupled to a B mode processor 26 and a Doppler processor 28. The B mode processor 26 employs amplitude detection for the imaging of structures in the body such as normal tissue, cysts, nerve fibers, and blood cells. B mode images of structure of the body may be formed in either the harmonic mode or the fundamental mode or a combination of both as described in U.S. Pat. No. 6,283,919 (Roundhill et al.) and U.S. Pat. No. 6,458,083 (Jago et al.) The Doppler processor processes temporally distinct signals from tissue and blood flow for the detection of motion of substances such as the flow of blood cells in the image field. The structural and motion signals produced by these processors are coupled to a scan converter 32 and a multiplanar reformatter 34, which produce image data of tissue structure, flow, or a combined image of both characteristics. The scan converter will convert echo signals with polar coordinates into image signals of the desired image format such as a sector image in Cartesian coordinates. The multiplanar reformatter will convert echoes which are received from points in a common plane in a volumetric region of the body into an ultrasonic image of that plane, as described in U.S. Pat. No. 6,443,896 (Detmer). A volume renderer (not shown) may also be employed to convert a the echo signals of a 3D data set into a projected 3D image as viewed from a given reference point as described in U.S. Pat. No. 6,530,885 (Entrekin et al.) The 2D or 3D images are coupled from the scan converter, multiplanar reformatter, and volume renderer (when used) to an image processor 30 for further enhancement, buffering and temporary storage for display on an image display 40.

A graphics processor 36 is also coupled to the image processor 30 which generates graphic overlays for displaying with the ultrasound images. These graphic overlays can contain standard identifying information such as patient name, date and time of the image, imaging parameters, and the like. For these purposes the graphics processor receives input from the user interface 38, such as a typed patient name. The user interface is also coupled to the transmit controller 18 to control the generation of ultrasound signals from the transducer array 10′ and hence the images produced by the transducer array and the ultrasound system. The user interface is also coupled to the multiplanar reformatter 34 for selection and control of a display of multiple multiplanar reformatted (MPR) images in accordance with the present invention as described below.

In accordance with the principles of the present invention, the probe 10 scans a volumetric region in front of the two dimensional array transducer and the echoes received from scanning this 3D volume are arranged into images of spatially aligned 2D image planes as illustrated by FIGS. 2a-2c. Such a spatial alignment in relation to a two dimensional array transducer 10′ is shown in FIG. 2c. In this drawing it is seen that the volume in front of (below in this illustration) the two dimensional array transducer 10′ is scanned by beams of ultrasound and the echoes received in response to the beam transmission are arranged to form 2D images of a sequence of adjacent image planes identified as a) through n). In this example the multiplanar reformatter 34 has formatted a spatial sequence of parallel non-intersecting planes. These image planes a) through n) are shown “edge-on” (orthogonal to the plane of the drawing) in FIG. 2a, from the perspective of the arrow 2a, 2b shown in association with FIG. 2c. The spacing and number of image planes are determined by the user and the type of array transducer used in the probe 10. There may be tens of image planes or scores of image planes, for instance. If the beams are closely spaced in the elevation dimension, closely spaced image planes can be formed and the number of planes over a given volume can be large. More widely spaced beams will produce more widely spaced image planes over the same dimension. The thickness of the image planes in the elevation dimension can be thin with tightly focused beams and the image planes can be slightly spaced apart from each other or contiguous. The image planes can also be overlapping in the thickness dimension as shown by the enlarged view to the left of FIG. 2a. In this example each image plane overlaps one-half of its adjacent image planes on each side, as shown by the brackets indicating the thickness of image planes a), b), and c). Image planes which overlap in thickness can be formed by overlapping “thick slice” images which are described in US Pat. pub. no. US2010/0168580 (Thiele).

FIG. 2b illustrates another sequence of image planes a) through n) which are scanned by a probe 10. In this example the non-intersecting planes are not perfectly parallel, but are slightly angled to diverge slightly from each other with increasing depth. This scan may be performed by steering the transmitted beams at the small angles offset from the orthogonal (normal) direction as this “edge-on” view of the image planes illustrates. These image planes will cover a wider field of view at increased depths than do the elevationally parallel planes of FIG. 2a, but with increased spacing between plane centers with increasing depth. When thick slice images are used in this technique the planes may be formed to overlap significantly in the near field but with decreasing overlap in the elevation direction with increasing depth.

The sequence of adjacent images, in either parallel planes or in angled image planes, can be formed in either of two ways in an embodiment of the present invention. One way is to direct the scanning beams in the desired image planes, then form each image from the echoes received from the beams which scan that plane. The other way is to acquire a 3D dataset of echo signals from points in the scanned volume, then use the multiplanar reformatter 34 to address and form an image of the echo data which is located in each desired plane. This addressing technique can form an image of a plane of any orientation through the 3D dataset by addressing and using only those datapoints which are located in a desired plane.

In accordance with the principles of the present invention, the adjacent image planes of the volumetric region formed by the multiplanar reformatter 34 are displayed in the sequence of their spatial order as illustrated by the ultrasound display of FIG. 3. As this example illustrates, the adjacent image planes are concurrently displayed. Each image plane is repetitively scanned in rapid succession so that each image in the display is a live, real-time image of its image plane. When the probe 10 is held against the body so that the target of an invasive procedure is within the field of view of the probe, the sequence of live images can be observed to guide a needle as it approaches and reaches the target site, and it is not necessary to maintain alignment of the needle with a single image plane. The progress of the needle insertion can be followed as it intersects successive image planes in the display. In the example of FIG. 3 the twelve images of adjacent image planes a) through n) show ultrasound images of the spine. The object of the procedure is to inject an anesthetic through a needle 70 into a nerve bundle 62 and to do so it is necessary to guide the insertion of the needle through the tissue of the body and cartilage 60 to reach the nerve bundle 62. A sequence of similar images may be seen in the case of a breast biopsy procedure, in which a fluid-filled cyst 60 surrounded by breast tissue contains a hard mass 62 at its core which it is desired to biopsy. The path of the needle as it approaches the nerve bundle 62 is not in alignment with a single image plane of the sequence. Instead, the needle 70 initially passes through image plane h) as it enters the body, then the angle of its insertion path passes through image plane g), and finally the needle reaches the target nerve bundle 62 in image plane f). In this example the needle will be seen to appear in image h), then image g), and then image f) in that order. When overlapping image planes are employed as shown in conjunction with FIG. 2a, adjacent images will contain some common image information. Thus, the same portion of the needle may appear in adjacent images. This is illustrated in FIG. 3, where some of the needle portion 70 of image g) is also seen in adjacent image f), and some of the needle portion of image g) is also seen in adjacent image h). This appearance of common image information will result in longer portions of the needle being visible in the images, affording improved needle visualization. The appearance of the needle 70 successively in these adjacent image planes gives the physician an intuitive sense of how the needle path is oriented with respect to the probe and hence where it is in the body and how it must be guided to reach the intended site of the procedure.

In a typical procedure a clinician will manipulate the probe 10 until the surgical site within the body is clearly in view, preferably in the center of the sequence of image planes, which would be images f) and g) in the example of FIG. 3. Alternately, the probe 10 may scan only a central image plane during this initial survey of the surgical site, then switch to the multiple MPR views when the procedure is started. The clinician will generally maneuver the probe in different orientations until the clinician finds what appears to be a good path for needle insertion in one of the images. This will generally be an intended needle insertion path which is aligned with one of the center images of the sequence. The clinician will usually prefer to follow the insertion of the needle in a single image, but the anatomy of the body may not readily accommodate this. The clinician will observe the position of the probe case or a marker on the case, which indicates the orientation of the image planes with respect to the probe position and, with or without the assistance of a needle guide, begin inserting the needle along the desired path. The needle may encounter harder and softer regions of tissue as it is inserted, causing the needle to vary from its intended path, even when the clinician is firmly guiding the needle. This change in direction can cause the needle to travel outside its single image plane in the elevation direction and into an adjacent plane. With a standard 2D imaging probe it is then necessary to adjust the position of the probe so that the entire needle, and particularly the needle tip is again in the image plane. It is also possible to move the two dimensional array probe 10 of the present example to realign the needle and its tip with a single image plane. But the present invention obviates this need to reposition the probe. Once an optimal acoustic window is found on the skin of the body the probe can be kept in that position. It is not necessary to move the probe from its acoustic window as the needle path changes because the needle tip will appear in the image of an adjacent image plane, as illustrated by FIG. 3. Thus, the clinician can maintain the stationary position of the probe against the body, or even have an assistant hold the probe in position, while the clinician focuses attention on the sequential image display (FIG. 3) and guidance of the needle. It is no longer necessary to continually try to maneuver the needle insertion or the probe to keep the needle in a single image plane.

The ability to maneuver the probe 10 with one hand while inserting the needle with another enables the clinician to optimize visualization of the needle in the images. The visualization of the needle in the ultrasound images can be poor if the angle of incidence of the ultrasound beams and the needle is not optimal. If the needle is inserted at a shallow angle such that it is almost parallel to the skin surface and plane of the transducer, the needle will be virtually a specular reflector, returning strong echoes from the nearly-orthogonal transmit beams. But when the needle is inserted at a steep angle, the steepness of the angle of incidence will cause energy of the beams to, in effect, glance off of the needle and travel away from the probe; very little energy is then reflected back to the transducer array. The needle may thus be difficult to clearly visualize in the images. But without the need to maintain alignment of the needle path and a single image plane, the probe may be reoriented or angularly directed beams used as shown in FIG. 2b to better optimize the angle of incident of the beams and needle path so that stronger echo signals will be reflected from the needle back to the transducer array and a sharper image of the needle formed.

When the probe and system form a large number of adjacent MPR images, it may not be possible to view all of the images on the display at the same time. The clinician may want to view the images in larger size, for instance, the example of twelve images in FIG. 3, to be able to observe the images better. Thus, images a) through n) of FIG. 3 may only be images of the twelve central image planes produced by the probe. There may be additional adjacent images before image a) and after image n) in the full sequence of adjacent images. In this case, the clinician will adjust a display control of the user interface 38 to select the group of adjacent images to show on the display. Generally, the clinician will roll the sequence up or down as the insertion of the needle progresses, keeping the image of the current position of the needle tip, image f) in the example of FIG. 3, in the middle of the currently displayed group of images. By doing this the next adjacent image plane reached by the needle tip will always be currently displayed, either in the center row, or on the row above or below in the sequence.

Another display format which may be employed is to use a single row or column of adjacent images rather than multiple rows of images as illustrated in FIG. 3. The row of images will be of a portion of the full image sequence, and the clinician will slide the row left or right with a user control to bring new image planes on one side or the other of the currently displayed group into display. With such a display the clinician will generally slide the displayed images left or right to maintain the image displaying the tip of the needle in the center image of the row. The center image may be shown in a larger size than the others in the row to improve visualization of the needle tip in that image. The use of a single row will generally require more manipulation of the user control to slide the display left or right than will the multiple row display of FIG. 3.

An implementation of the present invention will often be preferred by clinicians who are familiar with invasive procedures guided by 2D ultrasound and are unaccustomed to using 3D volumetric imaging for surgical guidance, as the procedure can be guided by use of a sequence of only 2D images as shown in FIG. 3. The clinician gains the advantage of 3D since multiple planes in a 3D volumetric region are scanned, but does not need to observe 3D volumetric imaged to guide the procedure, but only familiar 2D images.

1. An ultrasonic imaging system for guidance of insertion of an invasive device in a body comprising:
an ultrasound probe with a two dimensional array transducer which is adapted to steer beams of ultrasound over a volumetric region containing a surgical site to be accessed by the invasive device;
a beamformer which receives echoes returned in response to the beams for the formation of echo signals;
a multiplanar reformatter responsive to echoes received from the volumetric region for the formation of real-time images of a sequence of spatially adjacent image planes; and an ultrasound image display coupled to the multiplanar reformatter which is controlled to concurrently display a sequence of three or more real-time 2D images of spatially adjacent elevationally spaced image planes of the surgical site and surrounding anatomy in spatially adjacent order.

2. The ultrasonic imaging system of claim 1, wherein the image planes are spatially adjacent to each other in the elevation direction.

3. The ultrasonic imaging system of claim 2, wherein the image planes are spatially adjacent to each other in the thickness dimension.

4. The ultrasonic imaging system of claim 2, wherein the image planes are spatially contiguous with each other in the elevation direction.

5. The ultrasonic imaging system of claim 3, wherein adjacent image planes are spatially overlapping in the thickness dimension.

6. The ultrasonic imaging system of claim 3, wherein the images are thick slice images.

7. The ultrasonic imaging system of claim 1, wherein the planes of the spatially adjacent images are parallel to each other in the elevation direction.

8. The ultrasonic imaging system of claim 1, wherein the planes of the spatially adjacent images are angled with respect to each other in the elevation direction.

9. The ultrasonic imaging system of claim 1, wherein the image display displays multiple rows of images of spatially adjacent image planes, with an image plane of an image of each row being spatially adjacent to the image plane of an image of an adjacent row.

10. The ultrasonic imaging system of claim 1, wherein the multiplanar reformatter forms 2D images of a given number of adjacent image planes; and
wherein the image display displays a single row or column of images of adjacent image planes, wherein the number images in the single row or column is less than the given number.

11. The ultrasonic imaging system of claim 10, further comprising a user control;
wherein the image display is responsive to the user control to change the identity of the adjacent image planes which are displayed in the single row or column of images.

12. The ultrasonic imaging system of claim 10, wherein the row or column displays the image of a spatially first image plane at one end and a spatially last image plane at the other end of the row or column,
wherein the user control is adapted to display the image of an image plane which precedes the spatially first image plane or succeeds the spatially last image plane in the row or column.

13. The ultrasonic imaging system of claim 1, wherein the surrounding anatomy shown in one or more of the displayed images includes the insertion path of the invasive device.

14. The ultrasonic imaging system of claim 13, wherein the insertion path of the invasive device is shown in a plurality of spatially adjacent images.

15. The ultrasonic imaging system of claim 13, wherein a portion of the invasive device is shown in each of a plurality of images of adjacent image planes.

16. The ultrasonic imaging system of claim 15, wherein some of the portion of the invasive device shown on one image is also shown in the portion of the invasive device shown in the image of an adjacent image plane.
